(19) United States
(12) Patent Application Publication
(10) Pub. No.: US 2004/0244062 A1
(43) Pub. Date: Dec. 2, 2004

(54) Use of protein inhibitors as antithrombotic agents

(76) Inventors: Jill R. Crittenden, Cambridge, MA (US); Ann M. Graybiel, Lincoln, MA (US); David E. Housman, Newton, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/453,314

(22) Filed: Jun. 2, 2003

(51) Int. Cl. A01K 67/00; A61K 38/17; A61K 48/00

(52) U.S. Cl. 800/3; 800/8; 514/12; 514/44

(57) ABSTRACT

The invention relates generally to the use of protein inhibitors in the treatment of diseases and disorders associated with undesired thrombosis. Inhibiting activation of the protein encoded by the CalDAG-GEFI gene results in the reduction or prevention of blood clot formation. The invention provides methods and agents for inhibiting CalDAG-GEFI protein activity for use in antithrombotic therapy.

### FIELD OF THE INVENTION

The present invention relates generally to methods for treating or preventing diseases or disorders characterized by undesired thrombosis, and more particularly to methods for inhibiting CalDAG-GEFI (also known as RasGRP2) activity.

### BACKGROUND OF THE INVENTION

The formation of a blood clot within a blood vessel, a process called thrombosis, is a dangerous condition that can injure tissue and potentially lead to death. A variety of diseases and disorders are associated with thrombosis, including, for example, stroke, pulmonary embolus, myocardial infarction, restenosis and endothelial dysfunction. The process of thrombosis is dependent upon platelet aggregation, i.e., the adhesion of platelets to each other via a fibrinogen bridge. Platelet aggregation occurs only when fibrinogen, along with other proteins, bind to cell adhesion receptors, termed integrins, on the platelet surface. In order for fibrinogen to bind to the integrins, however, the integrins must be activated. One particular integrin known to mediate platelet aggregation is the GPIIb-IIIa integrin complex. GPIIb-IIIa exists on the surface of unstimulated platelets in inactive form, and when activated, becomes a receptor for fibrinogen, von Willerbrand Factor, and fibronectin.

The Rap1 protein has been implicated in the activation of GPIIb-IIIa, as well as in a number of other integrin-dependent processes. Inhibition of Rap1 protein function has been shown to interfere with the activation of integrins located on the platelet surface. Bos et al., Rap1 Signaling: Adhering to New Models, Nature Reviews 369-77 (May 2001). Rap proteins are members of the Ras small GTPase superfamily, and play an active role in the Ras/Raf-1(a serine/threonine kinase)/MAP kinase pathway by potentially inhibiting Ras signaling of the pathway, or, through B-Raf, can activate MAP kinase. A schematic diagram of the Ras signaling pathway is illustrated in FIG. 1.

Rap proteins comprise four isoforms, Rap1A, Rap1B, Rap2A and Rap2B, of which Rap1b and Rap2B are highly enriched in platelets. Rap proteins, like Ras proteins, cycle between inactive GDP-complexed and active GTP-complexed states. Guanine nucleotide exchange factors (GEFs) are required to activate Rap proteins by stimulating the release of GDP and the uptake of GTP. CalDAG-GEFI is a member of the RasGRP/CalDAG-GEF family of Ras GEFs, and has substrate specificity for Rap1 and Rap2. The nucleotide and peptide sequences for Mus musculus CalDAG-GEFI and Homo sapiens CalDAG-GEFI are reported in Kawasaki et al., 95 Proc. Natl. Acad. Sci. USA 13278-83 (1998), and Kawasaki et al., 282 Sci. 2275-79 (1998), the disclosures of both of which are incorporated by reference herein.

Various agents have been found to disrupt integrin activity and have been used to treat certain diseases or disorders associated with undesired thrombosis. Current products used in antithrombotic therapy, such as aspirin, dipyridamole and heparin, prevent the formation of blood clots by killing or removing platelets, but are either of low effectivity or present potential serious side effects, such as prolonged bleeding. Most of these products are administered intravenously, and/or on an emergency basis, and are intended to produce only short-term effects. Thus, there remains a need for identifying further means to treat or prevent diseases and disorders characterized by undesired thrombosis.

### SUMMARY OF THE INVENTION

The present invention is based upon the novel discovery that inhibiting CalDAG-GEFI function results in the reduction or prevention of platelet-mediated blood clot formation, and therefore the invention provides methods for treating or preventing diseases and disorders characterized by undesired thrombosis. Such diseases and disorders include, but are not limited to, acute coronary syndrome, myocardial infarction, unstable angina, refractory angina, restenosis, endothelial dysfunction, occlusive coronary thrombus occurring post-thrombolytic therapy or post-coronary angioplasty, thrombotically mediated cerebrovascular syndromes, embolic stroke, thrombotic stroke, transient ischemic attacks, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, vascularization of solid tumors and retinopathy. It is believed, without being limited to this theory, that the inhibition of CalDAG-GEFI function results in the inhibition of Rap1 function, which, in turn, interferes with the activation of, and fibrinogen binding to, the GPIIb-IIIa integrin, thereby reducing or preventing platelet aggregation and blood clot formation. The invention generally provides methods and agents for inhibiting CalDAG-GEFI activity for use in treating various diseases and disorders, and in particular, for antithrombotic therapy.

In one aspect, the invention relates to a method for treating or preventing a disease or disorder characterized by undesired thrombosis by administering to a patient an inhibitor of CalDAG-GEFI in an amount sufficient to reduce or prevent platelet-mediated blood clot formation. In one embodiment of the invention, the inhibitor interferes with the activation of CalDAG-GEFI by modulating calcium binding at a calcium-binding domain of CalDAG-GEFI. In another embodiment, the inhibitor interferes with the activation of CalDAG-GEFI by modulating diacylglycerol binding at a diacylglycerol-binding domain of the CalDAG-GEFI protein. In yet another embodiment, the inhibitor interferes with the binding of an effector molecule at a guanine nucleotide exchange enzymatic domain of CalDAG-GEFI. In a further embodiment, the inhibitor competitively binds to a substrate of CalDAG-GEFI.

In another aspect, the inhibitor interferes with the transcription of a CalDAG-GEFI gene. In another embodiment, the inhibitor interferes with translation of an mRNA sequence encoding a CalDAG-GEFI protein. In one aspect, the method further includes providing an amount of siRNA to the cell, and the siRNA comprises a sequence substantially complementary to at least a portion of the CalDAG-GEFI mRNA sequence. The amount is sufficient to reduce or eliminate translation of the mRNA in the cell. In certain embodiments, the siRNA can be duplexed or single-stranded. In another embodiment, the siRNA comprises a sequence having between about 20 and about 25 nucleotide bases.

In another aspect, the invention provides a cell line comprising at least one copy of recombinant nucleotide sequence encoding an inhibitor of CalDAG-GEFI. In another embodiment, the cell line comprises at least one copy of a partial deletion or a complete deletion of the CalDAG-GEFI gene. In one aspect, the nucleotide sequence is present in a viral vector. The viral vector may comprise an adenoviral vector such as a human adenovirus type 5 vector. The human adenovirus vector can also comprise a replication-deficient adenoviral vector.

In yet another aspect, the invention provides a non-human animal model for antithrombotic therapy. The non-human animal model comprises at least one copy of a recombinant nucleotide sequence encoding an inhibitor of CalDAG-GEFI. In another embodiment, the non-human animal model comprises at least one copy of a partial deletion or a complete deletion of the CalDAG-GEFI/RasGRP2 gene.

In yet another aspect, the invention generally comprises a non-human animal model for antithrombotic therapy, and the animal's genome comprises a modified copy of a CalDAG-GEFI gene. In one embodiment, the modified copy of a CalDAG-GEFI gene encodes a mutant CalDAG-GEFI protein.

Another aspect of the invention includes a non-human animal model for antithrombotic therapy wherein a genome of the animal, or an ancestor thereof, has been modified by at least one recombinant nucleotide sequence encoding an inhibitor of CalDAG-GEFI activity.

A further aspect of the invention relates to a method of identifying an antithrombotic agent. The method comprises performing an assay to determine an agent having an inhibitory effect on a CalDAG-GEFI activity.

### BRIEF DESCRIPTION OF THE DRAWINGS

FIG. 1 is a partial schematic diagram of a Rap signal transduction pathway.

FIG. 2 shows an illustrative diagram of the CalDAG-GEFI protein.

FIG. 3A shows a schematic diagram of a strategy for generating a CalDAG-GEFI “knockout” mouse according to an illustrative embodiment of the invention.

FIGS. 3B-3D shows the results of a strategy for generating a CalDAG-GEFI “knockout” mouse according to the illustrative embodiment of FIG. 3A.

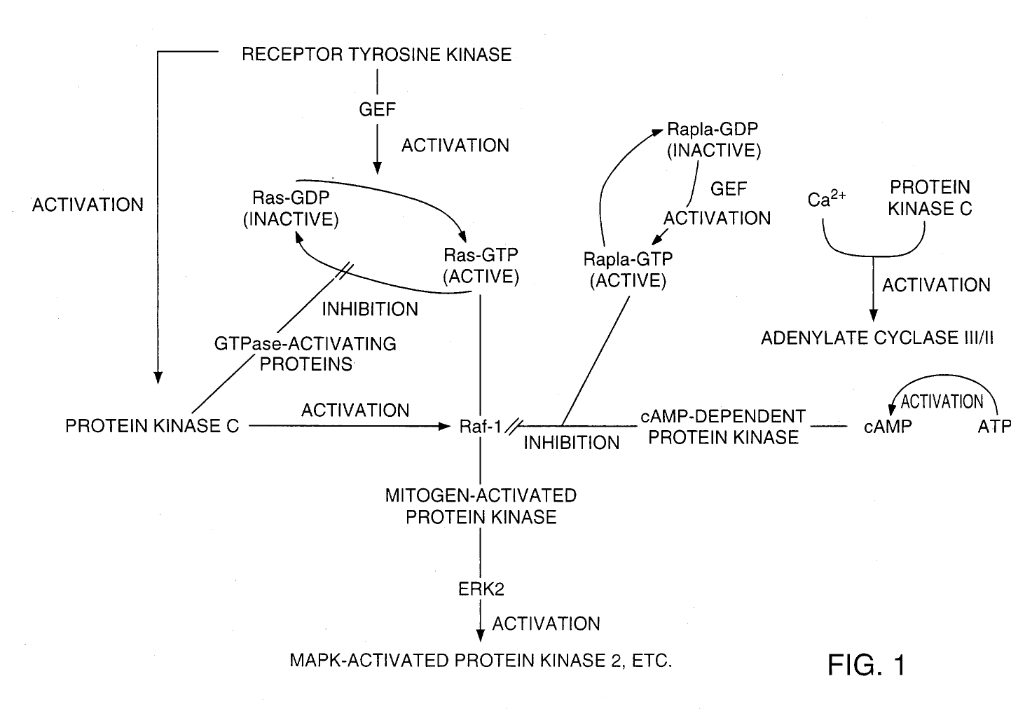

### DETAILED DESCRIPTION OF THE INVENTION

The present invention is based generally upon the inventors' novel discovery that inhibition of the CalDAG-GEFI protein results in the reduction or prevention of platelet-mediated blood clot formation, thereby serving as a means to treat or prevent diseases and disorders related to undesired thrombosis. Novel methods and agents of the present invention are based upon, but not limited to, (1) the administration of inhibitors of CalDAG-GEFI, including (i) administration of agents that modulate calcium binding at the calcium binding domain of a CalDAG-GEFI protein, (ii) administration of agents that modulate DAG binding at DAG binding domain of a CalDAG-GEFI protein, (iii) administration of agents that interfere with the binding of an effector molecule at a guanine nucleotide exchange enzymatic domain of a CalDAG-GEFI protein, (iv) administration of agents that interfere with the transcription of a CalDAG-GEFI gene, (v) administration of agents that interfere with the translation of an mRNA encoding a CalDAG-GEFI protein; and (vi) administration of agents that competitively bind with a substrate of CalDAG-GEFI; (2) gene therapy with a nucleic acid sequence encoding an inhibitor of CalDAG-GEFI; (3) gene therapy based upon antisense sequences to the CalDAG-GEFI gene or which “knock-out” the gene; and (4) gene therapy based upon siRNA sequences substantially complementary to a portion of an mRNA sequence encoding a CalDAG-GEFI protein. Methods and agents useful for inhibiting the activity and functional roles of the CalDAG-GEFI protein are described below, and may be utilized to treat such diseases or disorders.

The CalDAG-GEFI mammalian genes are represented by SEQ ID NOS: 1 and 3, as well as any allelic variants and heterospecific mammalian homologues. A murine CalDAG-GEFI cDNA sequence is disclosed herein as SEQ ID NO: 1, and a human CalDAG-GEFI cDNA sequence is disclosed herein as SEQ ID NO: 3. The CalDAG-GEFI gene, according to the current invention, primarily relates to a coding sequence, but can also include some or all of the flanking regulatory regions and/or introns, and specifically includes artificial or recombinant genes created from CDNA or genomic DNA, including recombinant genes based upon splice variants.

The CalDAG-GEFI protein, according to the current invention, includes allelic variants and heterospecific mammalian homologues. A murine CalDAG-GEFI protein sequence is disclosed herein as SEQ ID NO: 2, and a human CalDAG-GEFI protein sequence is disclosed herein as SEQ ID NO: 4. Splice variants are also embraced by the term CalDAG-GEFI protein as used herein. At least two splice forms of CalDAG-GEFI have been identified in humans, one that comprises 608 amino acids and is referred to as CalDAG-GEFI, and a longer splice form comprising 671 amino acids that is referred to as RasGRP2. Clyde-Smith, et al., Characterization of RasGRP2, a plasma membrane-targeted, dual specificity Ras/Rap exchange factor, J. Biol. Chem. 275 (41), 32260-32267 (2000). The protein may be produced by recombinant cells or organisms, may be substantially purified from natural tissues or cell lines, or may be synthesized chemically or enzymatically. Therefore, the CalDAG-GEFI protein, according to the invention, includes the protein in glycosylated, partially glycosylated, or unglycosylated forms, as well as in phosphorylated, partially phosphorylated, unphosphorylated, sulphated, partially sulphated, or unsulphated forms. It also includes allelic variants and other functional equivalents of the CalDAG-GEFI amino acid sequences, including biologically active proteolytic or other fragments.

The CalDAG-GEFI protein activates Rap1 and Rap2 and inhibits Ras-dependent activation of the Erk/MAP kinase cascade in 293T cells. Calcium ionophore and phorbol ester strongly and additively enhance Rap1 activation. Specific domains identified include structurally conserved GEF regions SCR1, SCR2, and SCR3, as shown in the following table.

TABLE 1

| | | | |
|---|---|---|---|
| Gene | SCR1 | SCR2 | SCR3 |

| | | | |
|---|---|---|---|
| hCa1DAG- | SEQ ID NO.3: | SEQ ID NO.3: | SEQ ID NO.3: |
| GEFI | 605-677 | 817-946 | 1053-1185 |
| | SEQ ID NO.4: | SEQ ID NO.4: | SEQ ID NO.4: |
| | 149-173 | 219-262 | 298-320 |

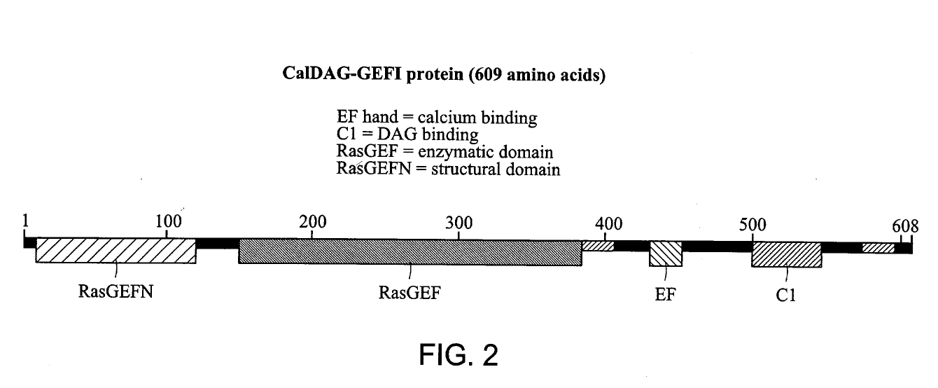

In addition, the EF hand (calcium-binding domains) and DAG-binding domains are shown in FIG. 2, and are identified in the following table:

TABLE 2

| | | |
|---|---|---|
| Gene | EF Hand Domain | DAG-Binding Domain |

| | | |
|---|---|---|
| hCa1DAG-GEFI | SEQ ID NO.3: | SEQ ID NO.3: |
| | 1456-1516 | 1652-1804 |
| | SEQ ID NO.4: | SEQ ID NO.4: |
| | 432-452 | 498-548 |

One aspect of the current invention generally provides a method for treating or preventing a disease or disorder in a patient by administering an inhibitor of CalDAG-GEFI to the patient in an amount and/or concentration sufficient to reduce or prevent platelet-mediated blood clot formation. The methods and agents of the invention are useful in (a) the treatment or prevention of any thrombotically mediated acute coronary syndrome including myocardial infarction, unstable angina, refractory angina, occlusive coronary thrombus occurring post-thrombolytic therapy or post-coronary angioplasty, (b) the treatment or prevention of any thrombotically mediated cerebrovascular syndrome including embolic stroke, thrombotic stroke or transient ischemic attacks, (c) the treatment or prevention of any thrombotic syndrome occurring in the venous system including deep venous thrombosis or pulmonary embolus occurring either spontaneously or in the setting of malignancy, surgery or trauma, thrombotic thrombocytopenic purpura, thromboangiitis obliterans, or thrombotic disease associated with heparin induced thrombocytopenia, (e) the treatment or prevention of thrombotic complications associated with extracorporeal circulation (e.g., renal dialysis, cardiopulmonary bypass or other oxygenation procedure, plasmapheresis), (f) coagulopathy and disseminated intravascular coagulation (g) the treatment or prevention of thrombotic complications associated with instrumentation (e.g., cardiac or other intravascular catheterization, intra-aortic balloon pump, coronary stent or cardiac valve), (h) those involved with the fitting of prosthetic devices, (i) vascularization of solid tumors and (j) retinopathy.

As used herein, an “inhibitor of CalDAG-GEFI” inhibits, prevents, decreases, or impedes, the normal activity of CalDAG-GEFI protein. An inhibitor can function by a means including, but not limited to: causing CalDAG-GEFI protein to be degraded, binding to CalDAG-GEFI protein such that it is incapable of being activated, binding to CalDAG-GEFI protein such that it is unable to interact with an effector molecule, binding to a substrate of CalDAG-GEFI such that CalDAG-GEFI is unable to bind thereto, inhibiting transcription of CalDAG-GEFI, and inhibiting translation of CalDAG-GEFI. Screening assays disclosed in this application, as well as those known to one skilled in the art, can be used to identify such inhibitors

Inhibitors suitable for use in the method of treatments disclosed herein include, but are not limited to, protein-based agents, carbohydrate-based agents, lipid-based agents, nucleic acid-based agents, natural agents, synthetically derived agents, anti-idiotypic antibodies and/or catalytic antibodies (or anitbody fragments thereof), ions, small molecules, organic agents or inorganic agents. It can be obtained, for example, from libraries of natural or synthetic agents, in particular from chemical or combinatorial libraries (i. e., libraries of agents that differ in sequence or size but that have the same building blocks) or by rational drug design.

In one embodiment, inhibitors of CalDAG-GEFI include polyclonal and monoclonal antibodies, including antibody fragments, Fab fragments, F(ab′)₂, and single chain antibody fragments, which selectively bind to CalDAG-GEFI, or to specific antigenic determinants of CalDAG-GEFI. The antibodies may be raised in mouse, rabbit, goat or other suitable animals, or may be produced recombinantly in cultured cells such as hybridoma cell lines.

Inhibitors of the invention may interfere with CalDAG-GEFI activity in any of a variety of ways. For example, the inhibitor may interfere with the activation of CalDAG-GEFI by modulating calcium binding at a calcium-binding domain (at the EF hand) of CalDAG-GEFI, or by modulating diacylglycerol (DAG) binding at a diacylglycerol-binding domain of the CalDAG-GEFI protein. In another embodiment, the inhibitor may interfere with the binding of an effector molecule at a guanine nucleotide exchange enzymatic domain of CalDAG-GEFI. In a further embodiment, the inhibitor competitively binds to a substrate of CalDAG-GEFI.

Alternatively, according to another embodiment of the invention, CalDAG-GEF activity may be inhibited through the use of small interfering RNAs (“siRNAs”). siRNAs are double-stranded RNA molecules that inhibit the expression of a gene with which they share homology and have been used as a tool to down regulate the expression of specific genes in a variety of cultured cells as well as in invertebrate animals. In one embodiment, the siRNA may be a “hairpin” or stem-loop RNA molecule, comprising a sense region, a loop region and an antisense region complementary to the sense region. In other embodiments, the siRNA comprises two distinct RNA molecules that are non-covalently associated to form a duplex. In one aspect of the invention, siRNAs comprising a sequence substantially complementary to at least a portion of an mRNA encoding CalDAG-GEFI is administered in an amount that reduces or eliminates translation of the mRNA, and inhibits CalDAG-GEFI protein activity. The siRNA can be duplexed or single-stranded, and can comprise a sequence of varying lengths. In one embodiment, the sequence comprises between about 20 and about 25 nucleotide bases.

The present invention also provides for a cell line, in which at least one copy of a CalDAG-GEFI gene is modified or deleted, or which comprises at least one copy of recombinant nucleotide sequence encoding an inhibitor of CalDAG-GEFI. Cells suitable for use in the methods of the invention include normal cells or spontaneously occurring variants of normal cells, or genetically engineered cells, and may be mammalian, invertebrate, plant, insect, fungal, yeast and bacterial cells. In certain embodiments, a cell of the present invention is transformed with at least one heterologous nucleic acid sequence.

Methods of producing appropriate vectors, transforming cells with those vectors, and identifying transformants are already well known in the art and are only briefly reviewed here (see, for example, Sambrook et al. (1989) Molecular Cloning: A Laboratory Manual, 2nd ed., Cold Spring Harbor Laboratory Press, Cold Spring Harbor, N.Y.).

Appropriate vectors can include cloning vectors and expression vectors of all types, including plasmids, phagemids, cosmids, episomes, and the like, as well as integration vectors. The vectors may also include various marker genes (e.g., antibiotic resistance or susceptibility genes) which are useful in identifying cells that have been successfully transformed therewith. Vectors may be introduced into the recipient or “host” cells by various methods, including, for example, calcium phosphate transfection, strontium phosphate transfection, DEAE dextran transfection, electroporation, lipofection (e.g., Dosper Liposomal transfection reagent, Boehringer Mannheim, Germany), microinjection, ballistic insertion on micro-beads, protoplast fusion, bacterial transfer, spheroplast fusion, or, for viral or phage vectors, by infection with the recombinant virus or phage. The recombinant construct transformed into cells suitable for use in the present invention can either remain on extra-chromosomal vectors or can be integrated into the cell genome.

Transformed cells may express the sequence of interest, or may be used only to propagate the sequence. Expression of a recombinant construct of the present invention in a cell can be accomplished using techniques known to those skilled in the art. Briefly, a nucleic acid molecule is inserted into an expression vector in such a manner that the nucleic acid molecule is operatively joined to a transcription control sequence in order to be capable of affecting either constitutive or regulated expression of the gene when the gene is transformed into a host cell. The phrase “recombinant molecule”, as used herein refers to a gene operatively linked to at least one transcription control sequence on an expression vector. The phrase “expression vector”, as used herein refers to a DNA or RNA vector that is capable of transforming a host cell, of replicating within the host cell, and of affecting expression of the operatively linked gene. Expression vectors are capable of replicating to either a high or low copy number depending on their inherent characteristics. Transcription control sequences, which can control the amount of protein produced, include sequences that control the initiation, elongation, and termination of transcription. Particularly important transcription control sequences are those which control transcription initiation, such as promoter and upstream activation sequences.

Construction of desired expression vectors can be performed by methods known to those skilled in the art and expression can be in eukaryotic or prokaryotic systems. Procaryotic systems typically used are bacterial strains including, but not limited to various strains of E. coli, various strains of bacilli or various species of Pseudomonas. In prokaryotic systems, plasmids are used that contain replication sites and control sequences derived from a species compatible with a host cell. Control sequences can include, but are not limited to promoters, operators, enhancers, and ribosome binding sites. Expression systems useful in eukaryotic host cells comprise promoters derived from appropriate eukaryotic genes. Useful mammalian promoters include early and late promoters from SV40; other viral promoters such as those derived from baculovirus, polyoma virus, adenovirus, bovine papilloma virus, avian sarcoma virus or cytomegalovirus; or collagenase promoters. Expression vectors include any vectors that function (i.e., direct gene expression) in recombinant cells of the present invention including bacterial, yeast, other fungal, insect, and mammalian cells. Particularly preferred expression vectors include promoters useful for expressing recombinant molecules in human cells.

An expression system can be constructed from any of the foregoing control elements operatively linked to nucleic acid sequences using methods known to those of skill in the art. (see, for example, Sambrook et al. (1989) Molecular Cloning: A Laboratory Manual, 2nd ed., Cold Spring Harbor Laboratory Press, Cold Spring Harbor, N.Y.).

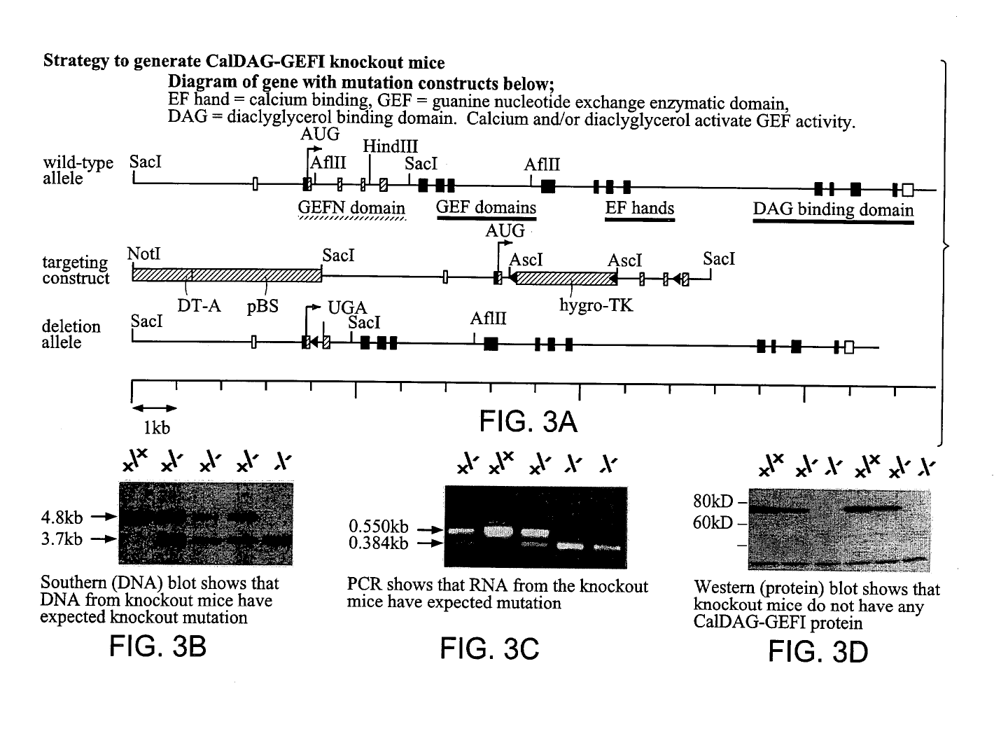

The current invention also provides for a non-human animal model for antithrombotic therapy, in which at least one copy of the animal's CalDAG-GEFI gene is modified or deleted, or which comprises a genome having a recombinant construct comprising a nucleotide sequence that encodes an inhibitor of CalDAG-GEFI. FIG. 3A includes an illustrative strategy used to generate an animal model in which the CalDAG-GEFI gene has been inactivated, or “knocked-out” in mice. FIGS. 3B-3D depict exemplary results of the illustrative strategy shown in FIG. 3A.

The non-human animal model may be used for studying disorders associated with thrombosis, for the screening of candidate pharmaceutical agents, for the creation of explanted mammalian cell cultures (e.g, neuronal, glial, organotypic or mixed cell cultures) in which mutant or wild type CalDAG-GEFI sequences are expressed or in which the CalDAG-GEFI gene has been inactivated, and for the evaluation of potential therapeutic interventions.

Species suitable for use as animal models in the present invention include, but are not limited to, rats, mice, hamsters, guinea pigs, rabbits, dogs,,cats, goats, sheep, pigs, and non-human primates (e.g., Rhesus monkeys, chimpanzees).

Various techniques for generating transgenic knock-out and knock-in animal models, as well as techniques for homologous recombination or gene targeting, are now widely accepted and practiced. See, for example, Hogan et al., Manipulating Mouse Embryo (1986). To create a transgene, the target sequence of interest (e.g., mutant CalDAG-GEFI sequence or an inhibitor of CalDAG-GEFI) is typically ligated into a cloning site located downstream of a promoter element which will regulate the expression of CalDAG-GEFI RNA. To delete a gene (knock-out), sequences that cause a stop in translation or transcription are targeted to the endogenous gene locus. More subtle changes are introduced to the endogenous locus to generate a knock-in, for example a mutant EF hand could be used to reduce calcium binding.

The invention also includes methods for identifying whether an agent is an inhibitor of CalDAG-GEFI expression or activity in accordance with the present invention. The assays may be performed in vitro using non-transformed cells, immortalized cell lines, or recombinant cell lines, or in vivo using the transgenic animal models described herein.

In particular, the assays may detect the presence of decreased expression of CalDAG-GEFI or other CalDAG-GEFI-related genes or proteins, on the basis of decreased mRNA expression (using, e.g., the nucleic acid probes) and protein expression (using, e.g., Western blotting techniques) or decreased levels of expression of a marker gene (e.g., β-galactosidase or luciferase) operably joined to a CalDAG-GEFI 5′ regulatory region in a recombinant construct.

Thus, for example, cell lines, such as 293T, can be engineered to express CalDAG-GEFI, Rap1, Elk1 transcription factor and an Elk1-responsive fluorescent reporter. Activation of CalDAG-GEFI activates Rap1, which then indirectly activates Elk1 to transcribe the fluorescent protein. Libraries of compounds (e.g., Chembridge) can be applied to the cells and ones that inhibit CalDAG-GEFI will inhibit fluorescence. To screen for specificity of CalDAG-GEFI inhibition rather than inhibition of Rap1 or some other member of the pathway, cells that express other activators of Rap1 along with the fluorescence reporter can be used. If the compound does not inhibit fluorescence of these cells, it can be deemed CalDAG-GEFI-specific.

Potential CalDAG-GEFI inhibitors identified by the above method may be further screened to determine which agents specifically interfere with GPIIb-IIIa integrin activity. The candidate agents can be added to a medium containing antibodies specific to activated GPIIb-IIIa (e.g.. the antibody Pac1) and cells known to express GPIIb-IIIa integrin receptors, such as platelets. Any agents that block the antibodies from binding can be considered to have an inhibitory effect on GPIIb-IIIa. GPIIb-IIIa inhibition can also be determined by adding a fluorescent label to fibrinogen and combining the labeled fibrinogen with both potential CalDAG-GEFI inhibitors and cells containing GPIIb-IIIa integrins. Those agents that interfere with GPIIb-IIIa activation and fibrinogen binding will result in the absence of fluorescence. Potential inhibitors can also be tested for provoking GPIIb-IIIa interference by combining labeled fibrinogen and cells known to contain GPIIb-IIIa integrins with substances known to activate GPIIb-IIIa, such as calcium and/or DAG. The cells will not exhibit fluorescence when agents that inhibit GPIIb-IIIa are added.

Other screening assays may be utilized to identify potential CalDAG-GEFI inhibitors. One may culture cells known to express CalDAG-GEFI protein and add to the culture medium one or more test agents. After allowing a sufficient period of time (e.g., 0-72 hours) for the agent to inhibit the expression of the CalDAG-GEFI protein, any change in the level of expression from an established baseline may be detected using any of the techniques described above or well known in the art. The cells can be from an immortalized cell line such as a human neuroblastoma, glioblastoma or a hybridoma cell line. Nucleic acid probes and/or antibodies can also be used to detect changes in the expression of CalDAG-GEFI, and thus, identification of agents as repressors of CalDAG-GEFI expression requires only routine experimentation.

Agents identified as inhibitors will have potential utility in modifying the function of CalDAG-GEFI or other CalDAG-GEF-related proteins in vivo. These agents may be further tested in the animal models disclosed herein to identify those agents having the most potent and least toxic in vivo effects for use in antithrombotic therapy. Moreover, small molecules having CalDAG-GEFI-binding activity may serve as “lead agents” for the further development of pharmaceuticals by, for example, subjecting the agents to sequential modifications, molecular modeling, and other routine procedures employed in rational drug design.

In addition, agents that bind to normal, mutant or both forms of the CalDAG-GEFI gene may have utility in antithrombotic treatments and diagnostics. Preferably, however, agents are identified which have a higher affinity of binding to normal CalDAG-GEFI, and which selectively or preferentially inhibit the function of the normal form. Such agents may be identified by comparing the CalDAG-GEFI binding affinities for all candidate agents. The effect of agents which bind to CalDAG-GEFI can be monitored either by direct monitoring of this binding (e.g., using the BIAcore assay, LKB Pharmacia, Sweden) or by indirect monitoring of binding by detecting, for example, a change in fluorescence, molecular weight, or concentration of either the binding agent or a CalDAG-GEFI component which comprises a CalDAG-GEFI polypeptide or portion thereof, either in a soluble phase or in a substrate-bound phase.

Further assays can be conducted to detect binding between a CalDAG-GEFI component and other moieties. Sequential assays in which agents are tested for the ability to bind to only the normal or only the mutant forms of the CalDAG-GEFI functional domains using mutant and normal CalDAG-GEFI components in the binding assays may be of particular utility. The CalDAG-GEFI component in these assays may be a complete normal or mutant form of the CalDAG-GEFI protein, or may be a specific domain of the CalDAG-GEFI protein. Particular functional domains of the CalDAG-GEFI protein, as described above, may be employed either as separate molecules or as part of a fusion protein. For example, to isolate proteins or agents that interact with these functional domains, screening may be carried out using fusion constructs and/or synthetic peptides corresponding to these regions. Obviously, various combinations of fusion proteins and functional domains of the CalDAG-GEFI protein are possible. In addition, the functional domains may be altered so as to aid in the assay by, for example, introducing into the functional domain a reactive group or amino acid residue (e.g., cysteine) which will facilitate immobilization of the domain on a substrate (e.g., using sulfhydryl reactions).

Methods for screening cellular lysates, tissue homogenates, or small molecule libraries for candidate CalDAG-GEFI-binding molecules are well known in the art, and in light of the present disclosure, may now be employed to identify agents which bind to normal CalDAG-GEFI components or which modulate CalDAG-GEFI activity as defined by non-specific measures (e.g., changes in intracellular Ca²⁺, GTP/GDP ratio) or by specific measures (e.g., changes in the expression of other downstream genes which can be monitored by differential display, 2D gel electrophoresis, differential hybridization, or SAGE methods).

Once identified by the methods described above, the inhibitors may be produced in quantities sufficient for pharmaceutical administration (e.g., μg or mg or greater quantities), and formulated in a pharmaceutically acceptable carrier (see, e.g., REMINGTON'S PHARMACEUTICAL SCIENCES, Gennaro, A., ed., Mack Pub., (1990)). The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17.sup.th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active agents. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation. A carrier (e.g., a pharmaceutically acceptable carrier) is generally preferred, but not necessary to administer the agent.

The inhibitor can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. The inhibitor can also be administered as at least one physiologically acceptable pharmaceutically-acceptable stereoisomers, hydrates, solvates, salts or prodrug derivatives. Optionally, the methods of this invention comprise administering such pharmaceutical compositions in combination with an additional therapeutic agent such as an antithrombotic, a thrombolytic agent or an anticoagulant, or any combination thereof.

The inhibitors used in the invention can be administered intravenously, parenterally, intramuscular, subcutaneously, orally, nasally, topically, by inhalation, by implant, by injection, or by suppository. The composition can be administered in a single dose or in more than one dose over a period of time to confer the desired effect.

As used herein, an “effective amount” of an agent is at least the minimum amount of an agent that is necessary to minimally achieve, and more preferably, optimally achieve, the desired effect (i.e., inhibition of CalDAG-GEFI activity). An effective amount for use in a given method can be readily determined by one skilled in the art without undue experimentation, depending upon the particular circumstances encountered (e.g., concentrations, cell type and number, and the like). A “therapeutically effective amount” of an inhibitor is the quantity of inhibitor which, after being administered to an individual or animal with undesired thrombosis, brings about an amelioration, slowing, arresting or reversion of the disease or disorder processes associated with the undesired thrombosis without causing unacceptable side-effects.

The skilled artisan will be able to determine the amount of inhibitor which is to be administered to a human or animal. The amount of inhibitor that is administered to an individual or animal will depend on a number of factors, including the general health, size, age, and sex of the individual or animal and the route of administration. It will also depend on the degree, location, severity and cause of the individual's or animal's undesired thrombosis. One of ordinary skill in the art will be able to determine the precise dosage according to these and other factors. A typical dosage might range from about 0.001 mg/kg to about 1000 mg/kg, preferably from about 0.01 mg/kg to about 100 mg/kg, and more preferably from about 0.10 mg/kg to about 20 mg/kg. Advantageously, the agents of this invention may be administered several times daily, and other dosage regimens may also be useful. Typically, about 0.5 to 500 mg of a compound or mixture of compounds of this invention, as the free acid or base form or as a pharmaceutically-acceptable salt, is agented with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

Furthermore, once identified by the methods described above, the candidate agents may also serve as lead agents in the design and development of new pharmaceuticals. For example, sequential modification of small molecules (e.g., amino acid residue replacement for peptides, or functional group replacement for peptide or non-peptide agents) is a standard approach in the pharmaceutical industry for the development of new pharmaceuticals. Such development generally proceeds from a lead agent which is shown to have at least some of the activity (e.g., ability to inhibit CalDAG-GEFI activity) of the desired pharmaceutical. In particular, when one or more agents having at least some activity of interest (e.g., modulation of CalDAG-GEFI protein activity) are identified, structural comparison of the molecules can greatly inform the skilled practitioner by suggesting portions of the lead agents which should be conserved and portions which may be varied in the design of new candidate agents. Thus, the present invention also provides a means of identifying lead agents that may be sequentially modified to produce new candidate agents for use in antithrombotic therapy. These new agents may also be tested for CalDAG-GEFI inhibition and for therapeutic efficacy and safety. This procedure may be iterated until agents having the desired therapeutic activity and/or efficacy are identified.

### EXAMPLE 1

### Identification of Inhibitors

To identify inhibitors of CalDAG-GEFI that could be used as antithrombotic or thrombolytic agents, chemical libraries in high-throughput screens followed by manual screens to test for activity in platelets are used. The first screen is designed to identify agents that inhibit CalDAG-GEFI signal transduction in a cell-based assay similar to that used by Kawasaki et al. (PNAS 1998). In this assay, the direct downstream effector of CalDAG-GEFI, Rap1, activates the transcription factor Elk1 to express a fluorescent reporter. Cell lines such as 293T are stabley transfected with vectors containing 1) CalDAG-GEF1, 2) Rap1, 3) Elk1 transcription factor and 4) an Elk1-responsive reporter with a short half-life (e.g., destabilized green fluorescent protein). Cells are grown in 96-well plates and assayed for reporter activity by an automatic plate reader at different time-points following agent application. Agents which reduce fluorescence are further analyzed.

Potential CalDAG-GEFI inhibitors from the first screen are applied to a second automated screen for specificity. Cell lines are stably transfected as above, but the CalDAG-GEFI construct is replaced with a vector carrying C3G or CalDAG-GEFIII, two proteins that activate Rap1. Agents that do not reduce fluorescence expression in this screen are likely to be CalDAG-GEFI specific and are further tested for functionality in platelets.

To test the ability of candidate agents to inhibit platelet aggregation, the candidate agents are applied to human platelets in the presence of activators that promote aggregation (ADP, thrombin and epinephrine). The extent of aggregation is detected turbidometrically by a commercial aggregometer; the amount of light absorbed is inversely proportional to the extent of aggregation.

Further controls are carried out to test the effective dosage, half-life, toxicity and cell permeability of promising agents. Additional screening of related agents are used to find the optimal inhibitor. Special attention will be given to agents such as calphostin C that have been shown to inhibit diacylglycerol activation of the DAG binding domains, including that in CalDAG-GEFII.

Finally, animal models are used to assay selected agents for function in vivo. Effective inhibitors are expected to increase bleeding time and reduce thrombus formation following oral or intravenous administration.

### EXAMPLE 2

### Modulation of Levels of CalDAG-GEFI In Vivo

CalDAG-GEFI levels in a cell may be modulated in vivo by altering the expression levels of endogenous CalDAG-GEFI gene, for example, by employing agents which affect transcription and/or translation of CalDAG-GEFI. For example, an inhibitor of the present invention may comprise a transcription factor that is capable of mediating the rate of CalDAG-GEFI transcription in a cell. The rate of transcription of the CalDAG-GEFI gene in a cell is not necessarily fixed and can change according to the needs of the cell in different conditions of growth. Such regulation of transcription can be mediated by proteins that, by binding to DNA near or within a promoter, can increase or decrease the rate at which RNA polymerase initiates RNA synthesis. Transcription rates can be mediated by proteins including transcription factors. Suitable transcription factors include, but are not limited to, at least a portion of a transcription factor.

In one embodiment, siRNAs can be used to modulate transcription of CalDAG-GEFI. The siRNAs can be transfected into cells where they target and cause degradation of mRNA homologous to the siRNAs. Various commercial siRNA products are available, such as those produced by Gene Therapy Systems, Inc., and may be utilized to practice the invention. In another embodiment, antisense molecules may be designed according to techniques known in the art and directed to CalDAG-GEFI to block the translation, post-transcriptional processing and/or transcription thereof (for example, see Selinfreund, R. H., et al., (1990) J. Cell Biol. 111, 2021-2028). Genes encoding antisense molecules may be transfected into cells to express the molecules in situ. Moreover, ribozymes may be used to achieve a similar effect, by selectively cleaving or blocking CalDAG-GEFI mRNA in vivo or in vitro and thus lowering the levels of CalDAG-GEFI.

The foregoing techniques lend themselves to methods of gene therapy wherein nucleic acids containing a CalDAG-GEFI-encoding sequence, or sequences containing ribozymes or antisense molecules directed against a CalDAG-GEFI-encoding nucleic acid, are transfected into an organism such that the CalDAG-GEFI, antisense molecule or ribozyme is produced in situ.

### EXAMPLE 3

### Modulation of Interactions at the Binding Site

In one aspect, the invention provides for the modulation of the interaction between CalDAG-GEFI and Rap1 or Rap2 at the level of the binding site. The Rap1 and Rap2 sequences are reported in Pizon et al.,. Human cDNAs rap1 and rap2 homologous to the Drosophila gene Dras3 encode proteins closely related to ras in the ‘effector’ region, Oncogene, August;3(2):201-4 (1998), the disclosure of which is incorporated by reference herein. Modulation may be performed in a number of ways, including, for example, (a) administering a molecular mimic of the binding site of the CalDAG-GEFI, thus competing for binding sites on Rap1 and/or Rap2 (collectively referred to as “Rap”), and reducing effective CalDAG-GEFI-Rap interaction; (b) administering a molecular mimic of the binding site of the Rap, thus competing for binding sites on the CalDAG-GEFI and reducing effective CalDAG-GEFI-Rap interaction; (c) administering an agent capable of causing an alteration in the binding site of the CalDAG-GEFI and/or the Rap, such as a conformational change, thereby affecting CalDAG-GEFI-Rap binding; (d) administering a modified Rap, or a modified CalDAG-GEFI wherein the binding site has been modified, for example, by selective mutagenesis, to provide for improved, reduced or altered specificity of binding; (e) administering a substance, other than a molecular mimic, which is capable of binding to the CalDAG-GEFI and/or Rap binding site, thus impeding CalDAG-GEFI-Rap interaction.

Molecular mimics may, for example, be peptides derived from the CalDAG-GEFI/Rap binding site of a CalDAG-GEFI or a Rap. Such peptides, for example, may be selected from the Rap binding domain comprising amino acids 150 to 383 (SEQ ID NO 4) of CalDAG-GEFI. Alternatively, the peptides may be selected from the corresponding binding domain on Rap. If appropriate, the entire 609 amino acid or 671 amino acid alternative splice form of CalDAG-GEFI may be used. However, advantageously, a smaller peptide is selected. Preferably, such a peptide may comprise 5 to 80, and more preferably 10 to 60, 20 to 50, 20 to 40 or 25 to 30 continuous amino acids from the Rap binding domain. Most preferably, the peptide comprises about 25 amino acids.

Moreover, the peptide may comprise non-continuous amino acids from the Rap domain; in other words, deletions, alterations or insertions may be performed in the domain to alter the properties of the peptide. Peptides comprising deletions and insertions are variants of the Rap binding domain. The variant provided by the present invention includes splice variants encoded by mRNA generated by alternative splicing of a primary transcript, amino acid mutants, glycosylation variants and other covalent derivatives of the Rap activating domain which retain the physiological and/or physical properties of Rap activating domain. Exemplary derivatives include molecules wherein the domain of the invention is covalently modified by substitution, chemical, enzymatic, or other appropriate means with a moiety other than a naturally occurring amino acid. Such a moiety may be a detectable moiety such as an enzyme or a radioisotope. Further included are naturally occurring variants of Rap activating domain found within a particular species, preferably a mammal. Such a variant may be encoded by a related gene of the same gene family, by an allelic variant of a particular gene, or represent an alternative splicing variant of a Rap gene.

Variants which retain common structural features can be fragments of the Rap activating domain. Fragments of the Rap activating domain comprise smaller polypeptides derived therefrom. Preferably, smaller polypeptides derived from the Rap activating domain according to the invention define a single feature which is characteristic of the Rap activating domain. Fragments may in theory be almost any size, as long as they retain the activity of the Rap activating domain described herein.

Derivatives of the Rap activating domain also comprise mutants thereof, which may contain amino acid deletions, additions or substitutions, subject to the requirement to maintain the activity of the Rap activating domain described herein. Thus, conservative amino acid substitutions may be made substantially without altering the nature of the Rap activating domain, as may truncations from the 5′ or 3′ ends. Deletions and substitutions may moreover be made to the fragments of the Rap activating domain comprised by the invention. Rap activating domain mutants may be produced from a DNA encoding the Rap activating domain which has been subjected to in vitro mutagenesis resulting, e.g., in an addition, exchange and/or deletion of one or more amino acids. For example, substitutional, deletional or insertional variants of the Rap activating domain can be prepared by recombinant methods and screened for immuno-crossreactivity with the native forms of the Rap activating domain.

The fragments, mutants and other derivatives of the Rap activating domain preferably retain substantial homology with the Rap activating domain. As used herein, “homology” means that the two entities share sufficient characteristics for the skilled person to determine that they are similar in origin and function. “Substantial homology”, as used herein, means that the two entities share more than 40% of the same characteristics, preferably more than 45% and most preferably 50% or more of the same characteristics. Homology, as used herein, can be based upon sequences retaining absolute sequence identity to the target sequence, as well as sequences that do not retain absolute sequence identity but comprise conservative amino acid substitutions while retaining functional aspects of the target sequence. Thus, the derivatives of the Rap activating domain preferably retain substantial homology with the Rap activating domain.

“Sequence identity”, as used herein, means that a sufficient percent of nucleotide residues in a candidate sequence are identical to the nucleotide residues in the target sequence such that a skilled person could conclude that the two sequences are similar. “Substantial sequence identity”, as used herein, means that the two entities share more than 40% sequence identity, preferably more than 45% sequence identity, and most preferably 50% or more sequence identity. To determine the percentage of sequence identity between a candidate fragment, mutant or other derivative and the target Rap activating domain, the candidate amino acid sequence and the target amino acid sequence are first aligned using the dynamic programming algorithm described in Smith and Waterman (1981), J. Mol. Biol. 147:195-197, in combination with the BLOSUM62 substitution matrix described in FIG. 2 of Henikoff and Henikoff (1992), PNAS 89:10915-10919. Computer programs performing alignments using the algorithm of Smith-Waterman and the BLOSUM62 matrix, such as the GCG program suite (Oxford Molecular Group, Oxford, England), are commercially available and widely used by those skilled in the art.

Once the alignment between the candidate and target sequence is made, a percent similarity score may be calculated. The individual amino acids of each sequence are compared sequentially according to their similarity to each other. If the value in the BLOSUM62 matrix corresponding to the two aligned amino acids is zero or a negative number, the pairwise similarity score is zero; otherwise the pairwise similarity score is 1.0. The raw similarity score is the sum of the pairwise similarity scores of the aligned amino acids. The raw score is then normalized by dividing it by the number of amino acids in the smaller of the candidate or reference sequences. The normalized raw score is the percent similarity. Alternatively, to calculate a percent identity, the aligned amino acids of each sequence are again compared sequentially. If the amino acids are non-identical, the pairwise identity score is zero; otherwise the pairwise identity score is 1.0. The raw identity score is the sum of the identical aligned amino acids. The raw score is then normalized by dividing it by the number of amino acids in the smaller of the candidate or reference sequences. The normalized raw score is the percent identity. Insertions and deletions are ignored for the purposes of calculating percent similarity and identity.

The invention therefore provides a pharmaceutical composition comprising a peptide which is a molecular mimic of the CalDAG-GEFI and/or Rap binding site. The composition may be formulated according to procedures well known to those skilled in the art, which are discussed for exemplification below.

### EXAMPLE 4

### Rational Drug Development

In another embodiment, the invention provides a peptide as defined above as a lead agent for the development of alternative agents, such as low molecular weight agents, which possess the same activity. This may be achieved in a number of ways; for example, the structure of the peptide may be modelled, for example, by using computer assisted modelling techniques, and low molecular weight agents designed such that they fit the binding site. In a particularly advantageous embodiment of the invention, the crystal structure of the peptide and/or a peptide/binding partner complex may be resolved. This will provide accurate information concerning the actual interaction between the peptide and Rap1/CalDAG-GEFI, allowing the precise design of a molecule designed to mimic this interaction at the precise binding site.

Alternatively, the peptide may be used in biological or biochemical approaches to drug discovery to identify a substance which is, for example, able to displace it from its binding site on CalDAG-GEFI or Rap1. For example, agents, preferably low molecular weight agents, may be screened in a method comprising the steps of:

forming a complex between a peptide according to the invention and its relevant binding partner;

incubating the complex with the agent to be screened, and monitoring for dissociation of the peptide/binding partner complex; and

selecting those agents which either favour or impede dissociation of the complex, compared to a control background.

In preferred embodiments, DNA encoding a peptide is ligated into a vector, and introduced into suitable host cells to produce transformed cell lines that express the peptide. The resulting cell lines can then be produced for reproducible qualitative and/or quantitative analysis of the effect(s) of potential drugs affecting Rap1 or CalDAG-GEFI function. Thus peptide expressing cells may be employed for the identification of agents, particularly small molecular weight agents, which modulate the function of CalDAG-GEFI or Rap1. Host cells expressing a peptide according to the invention are useful for drug screening and it is a further object of the present invention to provide a method for identifying agents which modulate the activity of a CalDAG-GEFI or Rap1, said method comprising exposing cells containing heterologous DNA encoding a peptide according to the invention, wherein said cells produce a functional CalDAG-GEFI and Rap1 which is a natural target therefor, to at least one agent or mixture of agents or signal whose ability to modulate the activity of the CalDAG-GEFI/Rap1 interaction is sought to be determined, and thereafter monitoring said cells for changes caused by said modulation. Such an assay enables the identification of modulators, such as agonists, antagonists and allosteric modulators of Rap1 or CalDAG-GEFI.

Cell-based screening assays can be designed by constructing cell lines in which the expression of a reporter protein, i.e., an easily assayable protein, such as beta-galactosidase, chloramphenicol acetyltransferase (CAT) or luciferase, is dependent on the activity of Rap1.

The present invention also provides a method to exogenously affect CalDAG-GEFI/Rap1 interactions occurring in cells. Rap1 and CalDAG-GEFI producing host cells, e.g., mammalian cells, can be contacted with a test agent, and the modulating effect(s) thereof can then be evaluated by comparing a Rap1 mediated response in the presence and absence of test agent.

Although embodiments of the invention have been described herein in detail, it will be understood by those skilled in the art that variations may be made thereto without departing from the spirit of the invention or the scope of the following claims.

What is claimed is:

1. A method for treating or preventing a disease or disorder in a patient, the method comprising
administering to the patient a amount of an inhibitor of a CalDAG-GEFI protein, said amount being sufficient to reduce or prevent platelet-mediated blood clot formation in the patient.

2. The method of claim 1 wherein said inhibitor interferes with activation of the CalDAG-GEFI protein by modulating calcium binding at a calcium binding domain of said CalDAG-GEFI protein.

3. The method of claim 1 wherein said inhibitor interferes with activation of the CalDAG-GEFI protein by modulating diacylglycerol binding at a diacylglycerol binding domain of said CalDAG-GEFI protein.

4. The method of claim 1 wherein said inhibitor interferes with the binding of an effector molecule at a guanine nucleotide exchange enzymatic domain of said CalDAG-GEFI protein.

5. The method of claim 1 wherein said disease or disorder is characterized by thrombosis.

6. The method of claim 1 wherein said disease or disorder is selected from the group consisting of acute coronary syndrome, myocardial infarction, unstable angina, refractory angina, restenosis, endothelial dysfunction, occlusive coronary thrombus occurring post-thrombolytic therapy or post-coronary angioplasty, thrombotically mediated cerebrovascular syndromes, embolic stroke, thrombotic stroke, transient ischemic attacks, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, vascularization of solid tumors and retinopathy.

7. The method of claim 1 wherein said administering step comprises administering a polypeptide.

8. The method of claim 7 wherein said administering step comprises administering a nucleotide having a sequence that encodes said inhibitor of CalDAG-GEFI protein.

9. The method of claim 8 wherein said nucleotide sequence is present in a viral vector.

10. The method of claim 9 wherein said viral vector is an adenoviral vector.

11. The method of claim 10 wherein said adenoviral vector is a human adenovirus type 5 vector.

12. The method of claim 11 wherein said human adenovirus vector is a replication-deficient adenoviral vector.

13. A method of reducing or eliminating translation of an mRNA sequence encoding a CalDAG-GEFI protein in a cell, the method comprising
providing an amount of an siRNA to the cell, said siRNA comprising a sequence substantially complementary to at least a portion of said mRNA, said amount being sufficient to reduce or eliminate translation of said mRNA in said cell.

14. The method of claim 13 wherein said siRNA is duplexed.

15. The method of claim 13 wherein said siRNA is single-stranded.

16. The method of claim 13 wherein said siRNA comprises a sequence having between about 20 and about 25 nucleotide bases.

17. A method for producing a non-human animal model for antithrombotic therapy comprising the steps of
providing a non-human animal; and
modifying at least one copy of a CalDAG-GEFI gene of said non-human animal.

18. The method of claim 17, wherein said modifying step comprises deleting at least one copy of a CalDAG-GEFI gene of said non-human animal.

19. A method for producing a non-human animal model for antithrombotic therapy comprising the steps of
providing a non-human animal; and
administering to said non-human animal a recombinant construct comprising a nucleotide sequence encoding an inhibitor of CalDAG-GEFI protein activity.

20. A non-human animal model for antithrombotic therapy wherein said animal's genome comprises at least one copy of a partial deletion or a complete deletion of the CalDAG-GEFI gene.

21. A non-human animal model for antithrombotic therapy wherein a genome of said animal, or an ancestor thereof, has been modified by at least one recombinant construct and wherein said recombinant construct comprises a nucleotide sequence encoding an inhibitor of a CalDAG-GEFI protein.

22. A method of identifying an antithrombotic agent comprising performing an assay to determine an agent having an inhibitory effect on a CalDAG-GEFI protein, thereby to determine an antithrombotic agent.

23. An antithrombotic composition comprising an inhibitor of a CalDAG-GEFI protein and a pharmaceutically acceptable carrier.

24. A cell line for antithrombotic therapy comprising at least one copy of a partial deletion or a complete deletion of the CalDAG-GEFI gene.

25. A cell line for antithrombotic therapy comprising at least one modified copy of a CalDAG-GEFI gene.


SEQUENCE LISTING (4 sequences)

SEQ ID NO: 1 - DNA, 2250 bp, Mus musculus
  CDS (166)..(1989): CalDAG-GEFI
cgaaggatcagaggctgagctggttcaagtgaacagaaggtctgggaggtgaactgcattcgggtttgcattctgaagtaaaggacttgggacaggggtacgaatcgagcactgtgggaggctctgagagtgtaacttgggtctagcccactggcaccggcagccatggcgagcactctggacctggacaagggttgcaccgtggaggagctgctccgtggctgtatcgaagcctttgatgactctggaaaggtgcgagatccacagctagtgcgcatgtttctcatgatgcacccctggtacataccttcctctcagctggcttcgaaactgctccacttctatcagcaatcccggaaggacaactccaattccctacaggtgaaaacctgtcacctggtcaggtactgggtctcagccttcccagcagagttcgacttgaacccagagctggctgaaccgatcaaggagctgaaggctctgttagaccaagaaggaaaccgcaggcacagcagcctcatcgacatcgagagtgtccccacctacaagtggaagcggcaggtgacccagcggaaccctgtggaacagaaaaagcgcaagatgtccctgttgtttgatcacttggagcctatggaactggcagaacatctcacctacttggagtatcggtccttctgcaagatcctgttccaggactatcacagctttgtgactcatggctgcactgtagacaatccggtcctggagcgattcatctccctcttcaacagtgtctctcagtgggtccaactcatgatcctcagcaagcccacagccacgcagcgggcgctggtcatcacacatttcgtgcatgtggcagagaagctgctgcagctgcagaacttcaacacgttgatggccgtcgtgggaggcctgagccacagctccatctcacgcctcaaggagacccacagccatgtcagccctgacaccatcaagctctgggaaggtctgacagaactagtgacagctactggcaactacagcaactaccggcgaaggctggcggcctgcgtgggcttccgctttcctatcctgggtgtgcacctcaaggatctagtggctctgcagctggctctgcctgactggctggacccaggtcggacccggctcaatggagccaagatgaggcagcttttcagcattctggaggagttggctatggtgaccagtcttcgaccaccagtgcaagccaaccccgacctgctgagtctgctcacggtgtccctggaccagtatcagacggaggatgagctgtatcagctctctctgcagcgagagccacgttccaagtcatcgcccaccagccccaccagctgcaccccgcctccccggccgcctgtgctggaagagtggacctcagttgccaagcctaagctggaccaagccttggtggccgagcacattgagaagatggtggagtctgtgttccggaactttgacgttgatggggacggtcacatctcccaggaggagttccagatcatccggggcaacttcccttatctcagcgcctttggggacttggaccagaaccaggatggctgcatcagccgggaggagatgatttcctacttcctgcgctccagctccgtgctgggaggccgcatgggcttcgtacacaacttccaggagagtaactcgctaaggccggtcgcctgccgacactgcaaagctctgatcctgggcatctacaagcagggcctcaaatgtagagcttgtggtgtgaactgccacaagcagtgcaaagaccgactgtcagtggaatgtcgccgccgcgcccagagtgtgagcctggagggctctgcaccctctccctcacccacacatacccaccatcgggccttcagcttctccctgcctcgcccaggcaggcgcagctctcggcctccagagatccgtgaagaggaggtgcagactgtggaagatggtgtgttcgacatccacttataagacgctgtgactatcaaggactcattcctgccttggagaaaagacttggagcagagcagggagccagggattctggggcaggaggttggggctgaaggtgggggaagttgaaggtggcatgcactgaaaaaaaggccagggctggtgtccctaaggttgtacagacttctgtgaatatttgtattttccagatggaataaaaaggcccgaataattaacctcgaaaaaaaaaaaaaaaaaaaaaaaaaaaaaaaaaaa

SEQ ID NO: 2 - protein, 608 aa, Mus musculus
MASTLDLDKGCTVEELLRGCIEAFDDSGKVRDPQLVRMFLMMHPWYIPSSQLASKLLHFYQQSRKDNSNSLQVKTCHLVRYWVSAFPAEFDLNPELAEPIKELKALLDQEGNRRHSSLIDIESVPTYKWKRQVTQRNPVEQKKRKMSLLFDHLEPMELAEHLTYLEYRSFCKILFQDYHSFVTHGCTVDNPVLERFISLFNSVSQWVQLMILSKPTATQRALVITHFVHVAEKLLQLQNFNTLMAVVGGLSHSSISRLKETHSHVSPDTIKLWEGLTELVTATGNYSNYRRRLAACVGFRFPILGVHLKDLVALQLALPDWLDPGRTRLNGAKMRQLFSILEELAMVTSLRPPVQANPDLLSLLTVSLDQYQTEDELYQLSLQREPRSKSSPTSPTSCTPPPRPPVLEEWTSVAKPKLDQALVAEHIEKMVESVFRNFDVDGDGHISQEEFQIIRGNFPYLSAFGDLDQNQDGCISREEMISYFLRSSSVLGGRMGFVHNFQESNSLRPVACRHCKALILGIYKQGLKCRACGVNCHKQCKDRLSVECRRRAQSVSLEGSAPSPSPTHTHHRAFSFSLPRPGRRSSRPPEIREEEVQTVEDGVFDIHL

SEQ ID NO: 3 - DNA, 2236 bp, Homo sapiens
  CDS (161)..(1987): CalDAG-GEFI
ggggactcaaggctggcctggctcaagtgaacagcacgtccaggaggcgacctcgtccgcgggtttgcattctggggtggacgagctgggggttcggtccgagcccggtgggaggctcccggagcgcagcctgggcccagcccaccccgcgccggcggccatggcaggcaccctggacctggacaagggctgcacggtggaggagctgctccgcgggtgcatcgaagccttcgatgactccgggaaggtgcgggacccgcagctggtgcgcatgttcctcatgatgcacccctggtacatcccctcctctcagctggcggccaagctgctccacatctaccaacaatcccggaaggacaactccaattccctgcaggtgaaaacgtgccacctggtcaggtactggatctccgccttcccagcggagtttgacttgaacccggagttggctgagcagatcaaggagctgaaggctctgctagaccaagaagggaaccgacggcacagcagcctaatcgacatagacagcgtccctacctacaagtggaagcggcaggtgactcagcggaaccctgtgggacagaaaaagcgcaagatgtccctgttgtttgaccacctggagcccatggagctggcggagcatctcacctacttggagtatcgctccttctgcaagatcctgtttcaggactatcacagtttcgtgactcatggctgcactgtggacaaccccgtcctggagcggttcatctccctcttcaacagcgtctcacagtgggtgcagctcatgatcctcagcaaacccacagccccgcagcgggccctggtcatcacacactttgtccacgtggcggagaagctgctacagctgcagaacttcaacacgctgatggcagtggtcgggggcctgagccacagctccatctcccgcctcaaggagacccacagccacgttagccctgagaccatcaagctctgggagggtctcacggaactagtgacggcgacaggcaactatggcaactaccggcgtcggctggcagcctgtgtgggcttccgcttcccgatcctgggtgtgcacctcaaggacctggtggccctgcagctggcactgcctgactggctggacccagcccggacccggctcaacggggccaagatgaagcagctctttagcatcctggaggagctggccatggtgaccagcctgcggccaccagtacaggccaaccccgacctgctgagcctgctcacggtgtctctggatcagtatcagacggaggatgagctgtaccagctgtccctgcagcgggagccgcgctccaagtcttcgccaaccagccccacgagttgcaccccaccaccccggcccccggtattggaggagtggacctcggctgccaaacccaagctggatcaggccctcgtggtggagcacatcgagaagatggtggagtctgtgttccggaactttgacgtcgatggggatggccacatctcacaggaagaattccagatcatccgtgggaacttcccttacctcagcgcctttggggacctcgaccagaaccaggatggctgcatcagcagggaggagatggtttcctatttcctgcgctccagctctgtgttgggggggcgcatgggcttcgtacacaacttccaggagagcaactccttgcgccccgtcgcctgccgccactgcaaagccctgatcctgggcatctacaagcagggcctcaaatgccgagcctgtggagtgaactgccacaagcagtgcaaggatcgcctgtcagttgagtgtcggcgcagggcccagagtgtgagcctggaggggtctgcaccctcaccctcacccatgcacagccaccatcaccgcgccttcagcttctctctgccccgccctggcaggcgaggctccaggcctccagagatccgtgaggaggaggtacagacggtggaggatggggtgtttgacatccacttgtaatagatgctgtggttggatcaaggactcattcctgccttggagaaaatacttcaaccagagcagggagcctgggggtgtcggggcaggaggctggggatgggggtgggatatgagggtggcatgcagctgagggcagggccagggctggtgtccctaaggttgtacagactcttgtgaatatttgtattttccagatggaataaaaaggcccgtgtaattaaccttcaaaaaaaaaaaaaaaaaaaa

SEQ ID NO: 4 - protein, 609 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL